(19) United States
(12) Patent Application Publication
(10) Pub. No.: US 2022/0054408 A1
(43) Pub. Date: Feb. 24, 2022

(54) COATED OCULAR IMPLANTS

(71) Applicant: RE-VANA THERAPEUTICS LTD, Belfast (GB)

(72) Inventors: Raghu Raj Singh Thakur, Belfast (GB); David Jones, Newtown abbey (GB); Rahul Sonawane, Belfast (GB); Yujing WANG, Belfast (GB); Karim Soliman, Hamilton (CA)

(21) Appl. No.: 17/414,959

(22) PCT Filed: Dec. 20, 2019
(86) PCT No.: PCT/EP2019/086834

§ 371 Date: Jun. 17, 2021

(30) Foreign Application Priority Data
Dec. 21, 2018 (EP) 18215413.8

(51) Int. Cl. A61K 9/00; A61K 31/5575; A61K 47/34

(52) U.S. Cl. CPC A61K 9/0051; A61K 47/34; A61K 31/5575

(57) ABSTRACT

The present invention relates to an ocular implant for the controlled release of a therapeutic agent or drug comprising: a) at least 0.1% w/w of a therapeutic agent; b) 5 to 95% w/w of a crosslinked polymer matrix; c) and 0.1 to 40% w/w of a biodegradable polymer selected from the group consisting of lactide/glycolide copolymer (including poly(lactide-co-glycolide) (PLGA)), poly (L-lactide) (PLA), polyhydroxyalkanoates, including polyhydroxybutyrate, polyglycolic acid (PGA), polycaprolactone (PCL), poly (DL-lactide) (PDL), poly (D-lactide), lactide/caprolactone copolymer, poly-L-lactide-co-caprolactone (PLC) and mixtures, copolymers, and block copolymers thereof; wherein the crosslinked polymer matrix is obtained by crosslinking a photopolymerizable composition selected from the group consisting of fragments or monomers of polyalkylene glycol mono-acrylate, polyalkylene glycol diacrylate, polyalkylene glycol mono-methacrylate and polyalkylene glycol dimethacrylate, and mixtures, copolymers, and block copolymers thereof, characterized in that the ocular implant is at least partially coated on its external surface with at least one coating layer selected from the group consisting of lactide/glycolide copolymer (including poly(lactide-co-glycolide) (PLGA)), poly (L-lactide) (PLA), polyhydroxyalkanoates, including polyhydroxybutyrate, polyglycolic acid (PGA), polycaprolactone (PCL), lactide/caprolactone copolymer, poly (DL-lactide) (PDL), poly (D-lactide), poly-L-lactide-co-caprolactone (PLC) and mixtures, copolymers, and block copolymers thereof; crosslinked fragments or monomers of polyalkylene glycol mono-acrylate, polyalkylene glycol diacrylate, polyalkylene glycol methacrylate and polyalkylene glycol dimethacrylate, and mixtures, copolymers, and block copolymers thereof.

### FIELD OF THE INVENTION

The present invention relates to coated ocular implants for the controlled release of a therapeutic agent or drug.

### BACKGROUND OF THE INVENTION

Chronic retinal diseases are the leading contributor to visual impairment and blindness worldwide. Loss of sight has a major personal impact on people's daily lives and has a profound economic impact on individuals, families, public health and society. The World Health Organization estimates that globally about 285 million people are visually impaired, of which 39 million are blind and 246 million have low vision. Diseases that originate in the posterior segment (PS) or back of the eye lead to permanent loss of vision if left untreated and account for the majority of blindness, such as in age-related macular degeneration (AMD), diabetic retinopathy (DR), diabetic macular edema (DME), cytomegalovirus (CMV) retinitis, retinitis pigmentosa, uveitis and glaucoma. The PS of the eye, which includes the retina, choroid, and vitreous body, is difficult to access due to the recessed location within the orbital cavity. Therefore, delivery of therapeutic agents to the PS of the eye has remained one of the most challenging tasks for pharmaceutical scientists and retina specialists.

Multiple approaches have been used to deliver therapeutic agents to the PS of the eye such as systemic, topical, periocular (or transscleral) and intravitreal. Topical (e.g. eye drops) and systemic (e.g. oral tablets) routes result in low or sub-therapeutic agent levels due to multiple ocular barriers, requiring administration of unnecessarily high concentrations of therapeutic agent that causes therapeutic agent-related toxicity and producing low treatment efficacy.

WO2017081154A1 discloses ocular compositions that can be administered to the eye in various forms to achieve controlled release of a therapeutic agent. These compositions can be used to form ocular implants by crosslinking the formulation either in situ after injecting it into the eye of a patient or can be preformed prior to injecting in the eye.

There is a need for alternative systems for ocular delivery of therapeutic agents.

### SUMMARY OF THE INVENTION

In a first aspect, the invention relates to a coated ocular implant that can be administered to the eye in various forms to achieve controlled release of a therapeutic agent or drug. Such ocular composition comprises:
a) at least 0.1% w/w of a therapeutic agent;
b) 5 to 95% w/w of a crosslinked polymer matrix;
c) and 0.1 to 40% w/w of a biodegradable polymer selected from the group consisting of lactide/glycolide copolymer (including poly(lactide-co-glycolide) (PLGA)), poly (L-lactide) (PLA), polyhydroxyalkanoates, including polyhydroxybutyrate, polyglycolic acid (PGA), polycaprolactone (PCL), poly (DL-lactide) (PDL), poly (D-lactide), lactide/caprolactone copolymer, poly-L-lactide-co-caprolactone (PLC) and mixtures, copolymers, and block copolymers thereof;

wherein the crosslinked polymer matrix is obtained by crosslinking a photopolymerizable composition selected from the group consisting of fragments or monomers of polyalkylene glycol mono-acrylate, polyalkylene glycol diacrylate, polyalkylene glycol mono-methacrylate and polyalkylene glycol dimethacrylate, and mixtures, copolymers, and block copolymers thereof,

characterized in that the ocular implant is at least partially coated on its external surface with at least one coating layer selected from the group consisting of lactide/glycolide copolymer (including poly(lactide-co-glycolide) (PLGA)), poly (L-lactide) (PLA), polyhydroxyalkanoates, including polyhydroxybutyrate, polyglycolic acid (PGA), polycaprolactone (PCL), lactide/caprolactone copolymer, poly (DL-lactide) (PDL), poly (D-lactide), poly-L-lactide-co-caprolactone (PLC) and mixtures, copolymers, and block copolymers thereof; crosslinked fragments or monomers of polyalkylene glycol mono-acrylate, polyalkylene glycol diacrylate, polyalkylene glycol methacrylate and polyalkylene glycol dimethacrylate, and mixtures, copolymers, and block copolymers thereof.

In a further aspect, the invention relates to a method of making the above ocular implant.

The present invention provides ocular implants that can be administered to the eye in various forms to achieve controlled release of a therapeutic agent The invention allows the flexibility to administer a range of small and large therapeutic molecules including proteins, peptides and gene therapeutics, and maintain their activity for a controlled period of time.

The ocular implants of the present invention enable to achieve long-term release by customizing and controlling the profile is function of the specific therapeutic agent(s) used and in accordance with the needs of the patient

The ocular implants of the present invention enable to suppress the so called “burst release” or “rapid initial release” effect, thus preventing that most of the therapeutic agent is released on the first day of the treatment. The patient is therefore never exposed to therapeutic agent doses which may exceed the maximum acceptable amount and, at the same time, the efficacy of the therapy is guaranteed by a sustainable release of the agent(s) over the entire period of treatment.

### DESCRIPTION OF THE FIGURES

FIG. 1 Shows the Scanning Electronic Microscopy (SEM) images of the implants DEX 1 and comparative example DEX 2.

FIG. 2 Shows the in vitro release of DEX from implants DEX1 and DEX2, expressed as percentage cumulative release (Mean±SD, n=3).

FIG. 3 Shows the in vitro release of TM from implants TM1 and TM2, expressed as percentage cumulative release (Mean±SD, n=3).

FIG. 4 Shows the in vitro drug release profile of FITC-dextran from implants D1 and CD1, expressed as percentage cumulative release (Mean±SD, n=3).

FIG. 5 Shows the in vitro drug release profile of LP from implants LP1, LP2, LPC1 and LPC2, expressed as percentage cumulative release (Mean±SD, n=3).

FIG. 6 Shows the in vitro drug release profile of LP from implants LPC1 and LPC3, expressed as percentage cumulative release (Mean±SD, n=3).

FIG. 7 Shows the in vitro drug release profile of LP from implants LPC1 and LPC4, expressed as percentage cumulative release (Mean±SD, n=3).

FIG. 8 Shows the in vitro drug release profile of LP from implants LP40 and LPC40, expressed as percentage cumulative release (Mean±SD, n=3).

### DETAILED DESCRIPTION OF THE INVENTION

As used herein, the term “% w/w” means the weight percentage of a given component over the total weight of the copolymer, the composition or the implant including such component, as the case may be.

As used herein, “biodegradable” is the chemical degradation by biological means. In some embodiments, the biodegradation is 100%, 98%, 90%, 85%, 80%, 60%, 50%, or 45% degradation of one or more of the compositions, monomers, oligomers, fragments, polymers, photoinitiators, solvents, co-solvents, or co-initiators.

As used herein “copolymer” is a mixture of two or more different types of monomer units. As used herein “block copolymer” is a mixture of two or more homopolymer subunits.

The therapeutic agent of the composition of the present invention can be selected from a wide range of small and large molecules. Exemplary therapeutic agents include, but are not limited to, polypeptides, nucleic acids, such as DNA, RNA, and siRNA, growth factors, steroid agents, antibody therapies, antimicrobial agents, antibiotics, antiretroviral therapeutic agents, anti-inflammatory compounds, antitumor agents, anti-angiogeneic agents, anti-VEGF (Vascular endothelial growth factor) agents, and chemotherapeutic agents.

In one embodiment, the therapeutic agent of the present invention includes, but is not limited to, ketorolac, naphazoline, lidocaine, bevacizumab, aflibercept, pegaptanib, brimonidine tartrate, dorzolamide, bromfenac sodium, azithromycin, rapamycin, bepotastine besilate, diclofenac, besifloxacin, cysteamine hydrochloride, fluocinolone acetonide, difluprednate, tasimelteon, ocriplasmin, enoxaparin sodium, ranibizumab, latanoprost, timolol maleate, bimatoprost, ofloxacin, cephazolin, phenylephrine, dexamethasone, triamcinolone acetonide, levofloxacin, cyclophosphamide, melphalan cyclosporine, methotrexate, azathioprine, travoprost, verteporfin, tafluprost, ketotifen fumarate, foscarnet, amphotericin B, fluconazole, voriconazole, ganciclovir, acyclovir, gatifloxacin, mitomycin-C, prednisolone, prednisone, vitamin (vitamin A, vitamin C, and vitamin E), zinc, copper, lutein, zeaxanthin or combinations thereof.

In another embodiment, the therapeutic agent of the present invention is dexamethasone, timolol maleate, brimonidine tartrate, triamcinolone acetonide, bromfenac sodium, latanoprost or mixtures thereof.

In one embodiment, the implants of the present invention can deliver bioactive agents, a large molecular weight therapeutic agent, such as, aflibercept, pegaptanib, or an antibody therapeutic, such as ranibizumab, bevacizumab, trastuzumab, rituximab, gentuzumab, ozagamicin, brolucizumab or cetuximab.

In some embodiments, the molecular weight of the therapeutic agent is greater than 200 Da, 500 Da, 1000 Da, 10 kDa, 30 kDa, 50 kDa, 75 kDa, 100 kDa, 150 kDa, 200 kDa.

According to other embodiments of the present invention, the therapeutic agent is present in an amount between 0.5 and 70% w/w, between 10 and 70% w/w, between 20 and 70% w/w, between 30 and 70% w/w, between 40 and 70%, between 5 and 50%, between 10 and 50% w/w, between 20 and 50% w/w, between 30 and 50% and between 40 and 50% of the total weight of the ocular implant.

The therapeutic agent can be used as such or in form of a solution wherein an amount of therapeutic agent is dissolved in a suitable solvent The therapeutic agent can also be freeze-dried or spray-dried before being used in the preparation of the ocular composition of the present invention in order to facilitate the incorporation of high concentrations of the therapeutic agent into the implant. The amount of the therapeutic agent to be dissolved depends on the final loading that the ocular composition or implant has to have. The choice of the solvent depends on the polarity of the therapeutic agent

According to an embodiment of the present invention, the solvent can be selected from water, dimethyl sulfoxide, decylmethyl sulfoxide, 2-pyrrolidone, 1-methyl-2-pyrrolidone, N-vinyl-pyrrolidine, N-Methyl-2-pyrrolidone, N-ethyl-pyrrolidone, glycerol formal, glycerol, polyethylene glycol, propylene glycol, benzyl alcohol, benzyl benzoate, ethyl benzoate, triacetin, triethyl citrate, dimethylformamide, dimethylacetamide and tetrahydrofuran.

In one embodiment, co-solvents may be used, and they can be selected from dichloromethane, tetrahydrofuran, ethyl acetate, acetone, dimethylformamide, acetonitrile, acetic acid, methanol, ethanol, isopropanol, glycofurol or butanol.

In case of hydrophilic therapeutic agents, the solvent may be an aqueous based solvent such as water or a phosphate buffered saline (PBS) solution.

According to another embodiment, the solvent may be selected from dimethyl sulfoxide, decylmethyl sulfoxide, 2-pyrrolidone, 1-methyl-2-pyrrolidne, N-methyl-2-pyrrolidone and glycerol formal.

Furthermore, the above described solvents and co-solvents can be used in the preparation of any of the implants of the invention, in combination with any of the other photopolymerizable compositions, biodegradable polymers, photoinitiators, pore forming agents, and co-initiators described herein.

In one embodiment, a solvent is used when the biodegradable polymer is PLGA, PCL, PLC, and/or PLA. In one embodiment the solvent is N-Methyl-2-pyrrolidone and N-Vinyl-2-pyrrolidine when the biodegradable polymer is PLGA, PCL, PLC, and/or PLA. In another embodiment, a solvent is used when the photopolymerizable composition is PEGDA.

The photopolymerizable fragments or monomers of the present invention can be used in any of the compositions and implants of the invention in combination with any of the other biodegradable polymers, therapeutic agents, photoinitiators, solvents, co-solvents, drug modulating agents and co-initiators described herein or known in the common general knowledge.

In one embodiment, the photopolymerizable composition of the invention can be biodegradable. In some embodiments the biodegradation takes place over 1 minute, 10 minutes, 20 minutes, 2 hours, 6 hours, 12 hours, 24 hours, 2 days, 5 days, 1 week, 1 month, 2 months, 5 months, 6 months, 8 months or 12 months. In some embodiments the biodegradation takes place between 1 month and 12 months, between 6 months and 12 months, or between 8 months and 12 months.

As used herein, the term “photopolymerizable composition” is a composition which can form a crosslinked polymer network upon exposure to light, in particular UV light. As used herein, photopolymerizable compositions include photopolymerizable monomers and oligomers (such as, dimers, trimers, and tetramers). The terms “oligomers” and “fragments” can be used interchangeably to mean between two and twenty monomers, optionally between two and ten monomers, further optionally between two and five monomers or between two and four monomers. A “photopolymerizable monomer” is a single unit of a photopolymerizable polymer that can bind chemically to other monomers to form a polymer.

Photopolymerizable compositions of the present invention can be crosslinked with UV radiation to form the crosslinked polymer matrix of the ocular implant of the present invention.

In one embodiment, the photopolymerizable composition is selected from the group consisting of fragments or monomers of polyalkylene glycol mono-acrylate, polyalkylene glycol diacrylate, polyalkylene glycol methacrylate, polyalkylene glycol dimethacrylate, and mixtures, copolymers, and block copolymers thereof.

In one embodiment, the photopolymerizable compositions are polyalkylene glycol diacrylate fragments or monomers incorporating diacrylate end units selected from the group comprising polyether fragments or monomers, polyester fragments or monomers, polycarbonate fragments or monomers or mixtures, copolymers, or block copolymers thereof.

In one embodiment, the photopolymerizable composition comprises monomers incorporating diacrylate end units, such as 4-arm or 8-arm PEG acrylate.

In another embodiment, the photopolymerizable composition is polyethylene glycol diacrylate, diethylene glycol diacrylate, polyethylene glycol dimethacrylate, diethylene glycol dimethacrylate, polypropylene glycol diacrylate, dipropylene glycol diacrylate, dipropylene glycol dimethacrylate, and polypropylene glycol dimethacrylate or mixtures, copolymers, or block copolymers thereof.

In another embodiment, the photopolymerizable composition is polyethylene glycol diacrylate (PEGDA), polyethylene glycol mono-acrylate (PEGMoA) or polyethylene glycol dimethacrylate (PEGDMA).

In yet another embodiment, the photopolymerizable composition is polyethylene glycol diacrylate (PEGDA).

In yet another embodiment, the photopolymerizable composition is polyethylene glycol methacrylate (PEGMA) or mixtures of PEGMA with other polyalkylene glycol mono-acrylates, diacrylates, methacrylates and/or dimethacrylates. In an embodiment, the polymerizable composition is a mixture of PEGDA, PEGMoA and/or PEGMA.

PEGDA is a synthetic polymer, available in different molecular weights. PEGDA is extremely amenable to mechanical, structural and chemical alteration and so resulting in hydrogels with variable properties in terms of drug delivery and other biomedical applications. PEGDA is formed through the functionalization of the end of each PEG molecule with an acrylate group. PEGDA is also non-toxic, eliciting only a minimal immunogenic response. PEGDA has double-bond containing acrylate end groups which show rapid polymerization when exposed to light in the presence of an appropriate initiator to produce a hydrogel network.

The average molecular weight of the photopolymerizable compositions of the present invention is typically between 100 and 300,000 Da, between 200 to 100,000 Da, between 200 to 50,000 Da, between 200 to 20,000 Da, between 200 to 10,000 Da, between 200 and 8,000 Da, between 200 and 5,000 Da, or between 200 and 1,000 Da.

It has been found, for the compositions and implants of the present invention, that an increase in molecular weight of the photopolymerizable compositions results in an increase in therapeutic agent release rate. Without wishing to be bound by theory, it is believed that photopolymerizable compositions with lower molecular weights have higher crosslinking density and therefore slower therapeutic agent release rates.

The photopolymerizable compositions of the present invention typically have viscosities between 0.1 to 7 dL/g, between 0.2 to 5 dL/g, or between 0.5 to 2 dL/g.

In an embodiment, the photopolymerizable composition is present in an amount between 10 and 90% w/w, between 10 and 75% w/w, between 20 and 75% w/w, between 30 and 75% w/w and between 30 and 60% w/w, between 40 and 60% w/w.

The biodegradable polymers of the present invention can be used in any of the compositions and implants of the invention in combination with any of the other photopolymerizable compositions, therapeutic agents, photoinitiators, solvents, co-solvents, therapeutic agent release modulating agents and co-initiators described herein or known in the common general knowledge.

In one embodiment of the present invention, the biodegradable polymers are aliphatic polyester-based polyurethanes, polylactides, polycaprolactones, polyorthoesters or mixtures, copolymers, or block copolymers thereof.

In another embodiment of the present invention, the biodegradable polymer is chitosan, poly(propylene fumarate), lactide/glycolide copolymer (including poly(lactide-co-glycolide) (PLGA)), poly (L-lactide) (PLA), polyglycolic acid (PGA), polycaprolactone (PCL), lactide/caprolactone copolymer (PLC), polyhydroxybutyrate, natural biodegradable polymers, such as collagen and hyaluronic acid, or mixtures, copolymers, or block copolymers thereof.

In another embodiment, the biodegradable polymer is selected from the group consisting of lactide/glycolide copolymer (including poly(lactide-co-glycolide) (PLGA)), poly (L-lactide) (PLA), polyhydroxyalkanoates, including polyhydroxybutyrate, polyglycolic acid (PGA), polycaprolactone (PCL), poly (DL-lactide) (PDL), poly (D-lactide), lactide/caprolactone copolymer, poly-L-lactide-co-caprolactone (PLC) and mixtures, copolymers, and block copolymers thereof.

In one embodiment, the biodegradable polymer is lactide/glycolide copolymer (including poly(lactide-co-glycolide) (PLGA)), poly (L-lactide) (PLA), poly(DL-lactide) (PDL), and lactide/caprolactone copolymer (PLC).

In a particular embodiment, the biodegradable polymer is poly(lactide-co-glycolide) (PLGA).

PLGA is typically prepared by polymerization of lactic acid and glycolic acid monomers. The glass transition temperatures (Tg) of PLGA copolymers are above physiological temperatures of 37° C., which imparts a moderately rigid chain configuration and therefore the mechanical strength at ambient temperatures. The use of PLGA in different lactide (LA) to glycolide (GA) ratio and molecular weight allows different drug release profiles. An increase in GA content will result in an increased water uptake of PLGA and therefore more rapid degradation. The degradation of PLGA with LA/GA 50/50 is typically between one and three months. In one embodiment, the molar ratio of lactic acid to glycolic acid in the PLGA is 90% lactic acid to 10% glycolic acid, 85% lactic acid to 15% glycolic acid, 75% lactic acid to 25% glycolic acid, 65% lactic acid to 35% glycolic acid, 50% lactic acid to 50% glycolic acid, 35% lactic acid to 65% glycolic acid, 25% lactic acid to 75% glycolic acid, 15% lactic acid to 85% glycolic acid, and 10% lactic acid to 90% glycolic acid.

In another embodiment, the biodegradable polymer is PCL, PLC, PLA, or mixtures, copolymers, or block copolymers thereof.

In an embodiment, the biodegradable polymer is present in an amount between 1 and 40% w/w, between 1 and 30% w/w, between 1 and 20% w/w, between 2 and 10% w/w and between 5 and 10% w/w.

In one embodiment of the present invention, the at least one coating layer comprises actide/glycolide copolymer (including poly(lactide-co-glycolide) (PLGA)), poly (DL-lactide) (PDL), poly (L-lactide) (PLA) and poly (D-lactide), and lactide/caprolactone copolymer, including poly-L-lactide-co-caprolactone (PLC) or combinations thereof.

In another embodiment, the at least one coating layer is poly (L-lactide) (PLA), poly (DL-lactide) (PDL) and lactide/caprolactone copolymer, including poly-L-lactide-co-caprolactone (PLC) or combinations thereof.

In another embodiment, the at least one coating layer is poly-L-lactide-co-caprolactone (PLC), poly (L-lactide) (PLA) or mixtures thereof.

In an embodiment, the at least one coating layer is a crosslinked photopolymerizable composition selected from the group consisting of polyethylene glycol diacrylate, diethylene glycol diacrylate, polyethylene glycol dimethacrylate, diethylene glycol dimethacrylate, polypropylene glycol diacrylate, dipropylene glycol diacrylate, dipropylene glycol dimethacrylate, and polypropylene glycol dimethacrylate.

In another embodiment, the at least one coating layer is crosslinked polyethylene glycol diacrylate (PEGDA).

In one embodiment, the ocular implant of the invention is at least partially coated on its external surface with at least two coating layers. In another embodiment, the ocular implant is at least partially coated on its external surface with at least three coating layers.

According to another embodiment, the ocular implant has a first and a second portion of external surface, wherein the first and second portion of the external surface are each coated with at least one coating layer independently selected from the group consisting of lactide/glycolide copolymer (including poly(lactide-co-glycolide) (PLGA)), poly (L-lactide) (PLA), polyhydroxyalkanoates, including polyhydroxybutyrate, polyglycolic acid (PGA), polycaprolactone (PCL), lactide/caprolactone copolymer, poly (DL-lactide) (PDL), poly (D-lactide), poly-L-lactide-co-caprolactone (PLC) and mixtures, copolymers, and block copolymers thereof; crosslinked fragments or monomers of polyalkylene glycol mono-acrylate, polyalkylene glycol diacrylate, polyalkylene glycol methacrylate and polyalkylene glycol dimethacrylate, and mixtures, copolymers, and block copolymers thereof.

In another embodiment, the ocular implant of the invention is coated on the totality of its external surface with at least one coating layer, at least two coating layers or at least three coating layers. The number of coating layers which are necessary depends on the viscosity of the solution of the coating material and, accordingly, the layer thickness that such solution can provide. The viscosity of the coating solution can be modified by changing, among others, the polymer concentration and the polymer molecular weight in order to optimize the release profile for each specific therapeutic agent

In one embodiment, the implant of the present invention comprises a release modulating agent A suitable release modulating agent may be selected in view of the specific therapeutic agent and composition of the implant, as well as the desired elution profile or release rate. The release modulating agent may be a naturally occurring agent or polymer or a synthetic agent or polymer.

All release modulating agents described herein can be used in any of the implants and compositions of the invention in combination with any of the other photopolymerizable compositions, biodegradable polymers, therapeutic agents, photoinitiators, solvents, co-solvents, and co-initiators described herein.

The release modulating agents may be present in amounts between 0.1 and 40% w/w, between 1 and 30% w/w, between 1 and 20% w/w, between 1 and 10% w/w, between 5 and 10% w/w.

Optionally, the release modulating agent alters water absorption into the implant matrix, thus controlling the release rate of the therapeutic agents and the implant degradation. In an embodiment, a suitable water absorption modulating agent is one or more polysaccharide like for example chitosan and cellulose based materials including hydroxypropyl methylcellulose (HPMC); hyaluronic acid; poloxamer; polyether like for example polyethylene glycol; gelatin; polyvinylpyrrolidone; polyvinyl alcohol and mixtures thereof. In one embodiment, suitable water absorption modulating agents are hydroxypropyl methylcellulose (HPMC) and polyethylene glycol (PEG).

In one embodiment, the release modulating agent is a pore-forming agent Optionally, it is lactose, maltose, glucose, mannitol, sodium chloride, magnesium carbonate, magnesium hydroxide, potassium chloride, sodium bicarbonate, ammonium bicarbonate, potassium bicarbonate, agarose or sucrose.

In another embodiment, the release modulating agent is a mixture of two or more modulating agents described above in order to provide more than one functionality to the ocular composition or implant of the present invention. Optionally, the release modulating agent is polyethylene glycol, hydroxypropyl methylcellulose (HPMC) or mixtures thereof.

Optionally, the at least one coating layer may be prepared in the presence of porosinogens so as to adjust coating porosity and thereby affect drug release. The pore size of the coating layer prepared by this porosinogen technique depends on the size of the porosinogens.

In another embodiment of the present invention, the ocular implant does not contain any release modulating agent

According to another embodiment, the at least one coating layer of the implant is porous.

According to another embodiment, the at least one coating layer has a thickness between 1 and 150 μm. In another embodiment, the at least one coating layer has a thickness between 15 and 40 μm.

In another embodiment, the at least one layer of the ocular implant of the present invention comprises at least some of the therapeutic agent. This can be the case, for example, if a second therapeutic agent has to be delivered from the same ocular implant. The second therapeutic agent may be present only in the coating while the first therapeutic agent only in the core of the implant, thus creating a differentiated release profile for the two agents. In another embodiment, the same therapeutic agent may be present both in the at least one coating layer and in the core of the implant, wherein the at least one coating layer is photo crosslinked to a different extent than the core of the implant. Accordingly, a differentiated release profile of the same therapeutic agent from the core and from the at least one coating layer of the implant is obtained.

The implants of the present invention can be of any desired shape such as but not limited to, rectangular, square, spherical cylindrical, circular, oval, films, dumbbell, rods and beads.

The implants of the present invention can have any desired size and can be, for example, in the macro, micro or nano particle size range.

In one embodiment of the present invention, the ocular implant is an implant which is less than 10 mm or less than 5 mm or less than 3 mm in one of the dimensions. In one embodiment, the implant is a rectangular implant of dimensions 10×5×0.5 mm. In one embodiment of the present invention, the ocular implant is a nanoparticle or a microparticle.

In one embodiment, the nanoparticle ocular implant is less than 1,000 nm, less than 900 nm, less than 750 nm, less than 500 nm, or less than 100 nm.

In one embodiment, the microparticle ocular implant is less than 1,000 μm, less than 900 μm, less than 750 μm, less than 500 μm, or less than 25 μm.

In one embodiment, the ocular implants of the present invention comprise the therapeutic agent in a concentration between 200 μg and 2000 μg per μm³, between 1000 μg and 2000 μg per μm³, between 1200 μg and 1800 μg per μm³, between 1200 μg and 1500 μg per μm³.

Another aspect of the present invention is a method of making an ocular implant as described above. The method comprises the subsequent steps of a) providing the therapeutic agent; b) obtaining an ocular composition by mixing the therapeutic agent with the polymerizable composition, the biodegradable polymer, a photoinitiator and optionally the release modulating agent; c) irradiating the ocular composition obtained under step b) with light at a wavelength between 200 and 550 nm for a period of time between 1 second and 60 minutes to form an uncoated ocular implant and d) coating at least a portion of the uncoated ocular implant external surface with at least one coating layer.

Optionally, under step b), the therapeutic agent is first mixed with the photopolymerizable composition and the so obtained mixture is mixed, in any order of addition, with the biodegradable polymer, the photoinitiator and optionally the release modulating agent Alternatively, the therapeutic agent is first mixed with a portion of the photopolymerizable composition and another portion of photopolymerizable composition is mixed with the biodegradable polymer, the photoinitiator and optionally the release modulating agent.

The photoinitiators described herein can be used in any of the compositions and implants of the present invention in combination with any of the other photopolymerizable compositions, biodegradable polymers, therapeutic agents, photoinitiators, solvents, co-solvents, and co-initiators described herein.

In certain embodiments, the photoinitiator is designed to work using light from 200 to 550 nm. In some embodiments, a photoinitiator is designed to work using UV light from 200 to 500 nm. In other embodiments, a photoinitiator is designed to work using UV light from 200 to 425 nm.

In certain embodiments, the light source may allow variation of the wavelength of light and/or the intensity of the light. Light sources useful in the present invention include, but are not limited to, lamps and fiber optics devices.

In one embodiment, the photoinitiator is a ketone (i.e. RCOR′). In one embodiment, the compound is an azo compound (i.e. compounds with a —N═N— group). In one embodiment, the photoinitiator is an acylphosphineoxide. In one embodiment, the photoinitiator is a sulfur containing compound. In one embodiment, the initiator is a quinone. In certain embodiments, a combination of photoinitiators is used.

In another embodiment, the photoinitiator may be selected from a hydroxyketone photoinitiator, an amino ketone photoinitiator, a hydroxy ketone/benzophenone photoinitiator, a benzyldimethyl ketal photoinitiator, a phenylglyoxylate photoinitiator, an acyl phosphine oxide photoinitiator, an acyl phosphine oxide/alpha hydroxy ketone photoinitiator, a benzophenone photoinitiator, a ribityl isoalloxazine photoinitiator, a peroxide photoinitiator, a persulfate photoinitiator or a phenylglyoxylate photoinitiator or any combination thereof. Optionally, the photoinitiator is 2-Hydroxy-4′-(2-hydroxyethoxy)-2-methylpropiophenone, 1-[4-(2-hydroxyethoxy)-phenyl]-2-hydroxy-2-methyl-1-propanone, 2,2-dimethoxy-2-phenylacetophenone (DMPA), diphenyl(2,4,6-trimethylbenzoyl) phosphine oxide (DPPO), or riboflavin. In another embodiment, the photoinitiator is benzoyl peroxide, 2,2″-azobisisobutyronitrile, dicumyl peroxide, lauroyl peroxide and/or camphorquinone.

In one embodiment, the compositions of the present invention further comprise a co-initiator. In one embodiment, the co-initiator is eosin Y, triethanolamine, camphorquinone, 1-vinyl-2 pyrrolidinone (NVP), eosin, dimethylaminobenzoate (DMAB), Irgacure® D-2959 (Sigma Aldrich, Basingstoke, UK), Irgacure® 907 (Sigma Aldrich, Basingstoke, UK), Irgacure® 651 (Sigma Aldrich, Basingstoke, UK), diphenyl (2,4,6-trimethylbenzoyl) phosphine oxide (DPPO/Darocur TPO) (Sigma Aldrich, Basingstoke, UK) or ethyl-4-N,N-dimethylaminobenzoate (4EDMAB). Optionally, the photoinitiator is riboflavin and the co-initiator is L-arginine.

In another embodiment, the therapeutic agent is first dissolved into a solvent to obtain a solution before the so obtained solution is mixed, under step b, with the polymerizable composition, the biodegradable polymer, the photoinitiator and optionally the release modulating agent.

The choice of the solvent which can be used according to the present invention depends on the polarity of the therapeutic agent

Optionally, the solvent can be selected from water, dimethyl sulfoxide, decylmethyl sulfoxide, 2-pyrrolidone, 1-methyl-2-pyrrolidne, N-vinyl-pyrrolidine, N-Methyl-2-pyrrolidone, N-ethyl-pyrrolidone, glycerol formal, glycerol, polyethylene glycol, propylene glycol, benzyl alcohol, benzyl benzoate, ethyl benzoate, triacetin, triethyl citrate, dimethylformamide, dimethylacetamide, acetonitrile, dichloromethane and tetrahydrofuran.

In one embodiment, co-solvents may be used and they can be selected from dichloromethane, tetrahydrofuran, ethyl acetate, acetone, dimethylformamide, acetonitrile, acetic acid, methanol, ethanol, isopropanol, glycofurol or butanol.

In case of hydrophilic therapeutic agents, the solvent may be an aqueous based solvent such as water or phosphate buffered saline (PBS) solution.

According to another embodiment, the solvent may be selected from dimethyl sulfoxide, decylmethyl sulfoxide, acetonitrile, 2-pyrrolidone, 1-methyl-2-pyrrolidne, N-methyl-2-pyrrolidone and glycerol formal.

Alternatively, the therapeutic agent is not dissolved into a solvent prior to mixing it with the other components. Accordingly, the therapeutic agent, the polymerizable composition, the biodegradable polymer, the photoinitiator and optionally the release modulating agent are mixed together in any order of addition. Alternatively, the therapeutic agent is first mixed with a portion of the photopolymerizable composition and another portion of photopolymerizable composition is mixed with the biodegradable polymer, the photoinitiator and optionally the release controlling agent.

In an embodiment, the ocular composition obtained under step b) is irradiated with light at a wavelength between 200 and 500 nm, between 200 and 490 nm, or between 200 to 425 nm, for a period of time between 1 second and 60 minutes, between 30 seconds and 30 minutes, between 2.5 minutes and 20 minutes, between 5 minutes and 10 minutes. In one embodiment, the crosslinking is for 3 seconds, 6 seconds, 9 seconds, 15 seconds, 30 seconds, 1, 2.5, 5, 10, 20 or 30 minutes.

In another embodiment, the uncoated ocular implant is coated under step d) on the totality of its external surface with at least one coating layer.

In one embodiment, the step d) of coating is performed by manual dipping, controlled dip-coating ultrasound coating, spray coating or 3D printing.

A further aspect of the present invention is an ocular implant obtainable by the method mentioned above.

In an embodiment of the present invention, the coated implant may be obtained by injecting an ocular composition comprising the therapeutic agent, the photopolymerizable composition, the biodegradable polymer, the photoinitiator and optionally a release modulating agent, into a preformed hollow tube of required dimensions made of the material of the at least one coating layer as described above. Accordingly, the coated implant of this embodiment has surface coating but not side coating.

In one embodiment, polymer molecular weight, type and copolymer ratio, drug type and loading, implant size, time and extent of UV crosslinking, amount and type of photoinitiator, release modulating agent, solvent and/or co-solvent can be altered to control the rate and extent of drug release. The alteration of these factors provides compositions of the invention that can be easily tailored to produce desired period of drug release to address specific clinical/patient needs in treating various ocular diseases.

The implants of the invention can be crosslinked prior to application in the eye to form an implant of desired shape and size (e.g. film, rod or nano/microparticles) that can be administered intraocularly to provide desired period of drug delivery, termed as Preformed Photocrosslinked Implants (PPcI).

The PPcIs of the present invention can be inserted in the eye, for example in the fornix, subconjunctively, intracameral, intrastromal/intracorneal, transsclerally/periocular, intrasclerally or intravitreally, subretinal, to treat the front of the eye or back of the eye diseases. The PPcIs can be fabricated in a variety of shapes including, but not limited to, rods, films, cylindrical or circular and sizes, including in the form of micro or nanoparticles.

In one embodiment, PPcI nano and microparticles are obtained by sonicating the mixture of therapeutic agent, photopolymerizable composition, biodegradable polymer, photoinitiator and, optionally, release modulating agent in an aqueous medium. In one embodiment, the aqueous medium is a combination of water and phosphate buffered saline (PBS). Irradiation can be applied during sonication i.e. sonicating the mixture under UV light or it can alternatively occur after the sonication step.

The PPcIs of the present invention have the advantage of high crosslink density and/or a tight polymer network structure which can be configured to control drug release and/or eliminate any burst release.

The PPcIs of the present invention can be fabricated to have a single and/or multiple layer which will enable loading of more than one drug or the same drug with different release profiles or rates.

The PPcIs of the invention comprise photopolymerizable polymers having a molecular weight typically between 100 and 300,000 Da, between 200 to 100,000 Da, between 200 to 50,000 Da, between 200 to 20,000 Da, or between 200 to 10,000 Da.

In one embodiment, the present invention is a PLGA/PEGDA PPcI.

In one embodiment, the biodegradable polymer is essentially contained within a matrix of the photopolymerizable composition. Optionally, the biodegradable polymer is essentially contained within a matrix of the photopolymerizable composition that forms a gel upon mixing. In one embodiment the photopolymerizable polymer is crosslinked in presence of a photoinitiator and the biodegradable polymer and therapeutic agent(s). In one embodiment, the biodegradable polymer is hydrophobic in nature and the photopolymerizable polymer is hydrophilic in nature. In one embodiment, the degree of crosslinking of the composite implant will govern the rate and extent of release of the therapeutic agent(s).

In the implants of the present invention, varying the UV crosslinking time can control the rate of and duration of drug release. In some embodiments, an increase in UV crosslinking times causes a decrease in drug release. Additionally, varying the concentration of the photoinitiator can control the rate and duration of drug release. Furthermore, varying both the UV crosslinking time and the concentration of photoinitiator can control the rate and duration of drug release. In one embodiment, decreasing the concentration of the biodegradable polymer (such as PLGA) increases the drug release rate. In one embodiment, adding a pore-forming agent (e.g. MgCO₃), increases the drug release rate. In one embodiment, higher UV crosslinking time and higher concentration of photoinitiator can sustain the drug release for longer periods of time. In one embodiment, the drug release can be sustained for a period of greater than 1 day, 2 days, 1 week, 1 month, 2 months, 3 months, or 6 months.

In some embodiments, the slow degradation rate of the PPcIs of the present invention provide protection of the sensitive molecules such as peptides and proteins.

In one embodiment, the present invention is a PPcI with high crosslinking density that significantly slows drug diffusion.

Any of the implants and compositions described herein are suitable for use in any of the methods of the invention described herein.

In one embodiment, the present invention is a method of treating a disease or disorder of the eye in a subject in need thereof, comprising administering a composition or implant of the present invention to an ocular area of the subject.

In one embodiment, the present invention is a composition or implant of the present invention for use in treating a disease or disorder of the eye in a subject in need thereof.

As used herein, an “ocular area” is an area inside, outside or adjacent to the eye of the subject In one embodiment, the ocular area is the sclera (intrascleral), outside the sclera (transscleral), the vitreous body, the choroid, the cornea, the stroma, intracameral, the aqueous humor, the lens, the fornix, or the optic nerve.

In one embodiment, the compositions and implants can be administered by injection, including, intravitreal, subconjunctival, peribulbar, subtenon or retrobulbar injections and cornea.

In some embodiments, the implants are administered via a surgical procedure. In some embodiments, the implants are secured in place, after surgical implantation, via an adhesive or sutures.

The term “subject” refers to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In another embodiment, the subject is a “human”.

As used herein, the terms “treat”, “treatment” and “treating” refer to therapeutic treatments includes the reduction or amelioration of the progression, severity and/or duration of a disease, disorder or condition, or the amelioration of one or more symptoms (specifically, one or more discernible symptoms) of a disease, disorder or condition, resulting from the administration of the compositions or implant of the invention. In specific embodiments, the therapeutic treatment includes the amelioration of at least one measurable physical parameter of a disease, disorder or condition. In other embodiments the therapeutic treatment includes the inhibition of the progression of a condition, either physically by, e.g., stabilization of a discernible symptom, physiologically by, e.g., stabilization of a physical parameter, or both. In other embodiments the therapeutic treatment includes the reduction or stabilization of a disease, disorder or condition.

In one embodiment, the disease, or disorder is pain, inflammation, cataracts, allergies, age-related macular degeneration (AMD), diabetic retinopathy (DR), macular edema, diabetic macular edema (DME), cytomegalovirus (CMV), retinitis, retinitis pigmentosa, uveitis, dry-eye syndrome, keratitis, glaucoma, blepharitis, blephariconjunctivtis, ocular hypertension, conjunctivitis, cystinosis, vitreomacular adhesion, corneal neovascularisation, corneal ulcers and post-surgical ocular inflammations/wound healing.

The following list of numbered items are embodiments comprised by the present invention:
1. An ocular implant comprising:
   a) at least 0.1% w/w of a therapeutic agent;
   b) 5 to 95% w/w of a crosslinked polymer matrix;
      and 0.1 to 40% w/w of a biodegradable polymer selected from the group consisting of lactide/glycolide copolymer (including poly(lactide-co-glycolide) (PLGA)), poly (L-lactide) (PLA), polyhydroxyalkanoates, including polyhydroxybutyrate, polyglycolic acid (PGA), polycaprolactone (PCL), poly (DL-lactide) (PDL), poly (D-lactide), lactide/caprolactone copolymer, poly-L-lactide-co-caprolactone (PLC) and mixtures, copolymers, and block copolymers thereof;
   wherein the crosslinked polymer matrix is obtained by crosslinking a photopolymerizable composition selected from the group consisting of fragments or monomers of polyalkylene glycol mono-acrylate, polyalkylene glycol diacrylate, polyalkylene glycol mono-methacrylate and polyalkylene glycol dimethacrylate, and mixtures, copolymers, and block copolymers thereof,
   characterized in that the ocular implant is at least partially coated on its external surface with at least one coating layer selected from the group consisting of lactide/glycolide copolymer (including poly(lactide-co-glycolide) (PLGA)), poly (L-lactide) (PLA), polyhydroxyalkanoates, including polyhydroxybutyrate, polyglycolic acid (PGA), polycaprolactone (PCL), lactide/caprolactone copolymer, poly (DL-lactide) (PDL), poly (D-lactide), poly-L-lactide-co-caprolactone (PLC) and mixtures, copolymers, and block copolymers thereof; crosslinked fragments or monomers of polyalkylene glycol mono-acrylate, polyalkylene glycol diacrylate, polyalkylene glycol methacrylate and polyalkylene glycol dimethacrylate, and mixtures, copolymers, and block copolymers thereof.
2. The ocular implant according to embodiment 1 or 2, wherein the therapeutic agent is present in an amount between 0.5 and 70% w/w.
3. The ocular implant according to embodiment 2, wherein the therapeutic agent is present in an amount between 10 and 50% w/w.
4. The ocular implant according to any preceding embodiment, wherein the therapeutic agent is present in an amount between 20 and 50% w/w.
5. The ocular implant according to any preceding embodiment, wherein the photopolymerizable composition is selected from the group consisting of fragments or monomers of polyalkylene glycol diacrylate, polyalkylene glycol dimethacrylate, and mixtures, copolymers, and block copolymers thereof.
6. The ocular implant according to any preceding embodiment, wherein the photopolymerizable composition is selected from the group consisting of polyethylene glycol diacrylate, diethylene glycol diacrylate, polyethylene glycol dimethacrylate, diethylene glycol dimethacrylate, polypropylene glycol diacrylate, dipropylene glycol diacrylate, dipropylene glycol dimethacrylate, and polypropylene glycol dimethacrylate.
7. The ocular implant according to embodiment 6, wherein the photopolymerizable composition is polyethylene glycol diacrylate (PEGDA).
8. The ocular implant according to any preceding embodiment, wherein the biodegradable polymer is present in an amount between 1 and 30% (w/w).
9. The ocular implant according to any preceding embodiment, wherein the biodegradable polymer is lactide/glycolide copolymer (including poly(lactide-co-glycolide) (PLGA)), poly (L-lactide) (PLA), poly(DL-lactide) (PDL), and lactide/caprolactone copolymer (PLC).
10. The ocular implant according to embodiment 9, wherein the biodegradable polymer is lactide/glycolide copolymer, including poly(lactide-co-glycolide) (PLGA).
11. The ocular implant according to any preceding embodiment, wherein the at least one coating layer is poly (L-lactide) (PLA), poly (DL-lactide) (PDL), poly-L-lactide-co-caprolactone (PLC) and combinations thereof.
12. The ocular implant according to embodiment 11, wherein the at least one coating layer is poly-L-lactide-co-caprolactone (PLC), poly(L-lactide) (PLA) or mixtures thereof.
13. The ocular implant according to any embodiment 1 to 10, wherein the at least one coating layer is a crosslinked photopolymerizable composition selected from the group consisting of polyethylene glycol mono-/di-acrylate, diethylene glycol diacrylate, polyethylene glycol dimethacrylate, diethylene glycol dimethacrylate, polypropylene glycol diacrylate, dipropylene glycol diacrylate, dipropylene glycol dimethacrylate, and polypropylene glycol dimethacrylate.
14. The ocular implant according to embodiment 13, wherein the at least one coating layer is crosslinked polyethylene glycol diacrylate (PEGDA).
15. The ocular implant according to any preceding embodiment, wherein it is at least partially coated on its external surface with at least two coating layers.
16. The ocular implant according to any preceding embodiment, wherein it is at least partially coated on its external surface with at least three coating layers.
17. The ocular implant according to any preceding embodiment, wherein the implant is coated on the totality of its external surface with at least one coating layer.
18. The ocular implant according to embodiment 17, wherein the implant is coated on the totality of its external surface with at least three coating layers.
19. The ocular implant according to any preceding embodiment, having a first and a second portion of external surface, wherein the first and second portion of the external surface are each coated with at least one coating layer independently selected from the group consisting of lactide/glycolide copolymer (including poly(lactide-co-glycolide) (PLGA)), poly (L-lactide) (PLA), polyhydroxyalkanoates, including polyhydroxybutyrate, polyglycolic acid (PGA), polycaprolactone (PCL), lactide/caprolactone copolymer, poly (DL-lactide) (PDL), poly (D-lactide), poly-L-lactide-co-caprolactone (PLC) and mixtures, copolymers, and block copolymers thereof; crosslinked fragments or monomers of polyalkylene glycol mono-acrylate, polyalkylene glycol diacrylate, polyalkylene glycol methacrylate and polyalkylene glycol dimethacrylate, and mixtures, copolymers, and block copolymers thereof.
20. The ocular implant according to any preceding embodiment, further comprising a release modulating agent.
21. The ocular implant according to embodiment 20, wherein the release modulating agent is selected from polyethylene glycol, hydroxypropyl methylcellulose (HPMC), maltose, glucose, agarose, mannitol, gelatin, sodium chloride, magnesium carbonate, magnesium hydroxide, potassium chloride, sodium bicarbonate, potassium bicarbonate and sucrose.
22. The ocular implant according to embodiment 21, wherein the release modulating agent is polyethylene glycol, hydroxypropyl methylcellulose (HPMC) or mixtures thereof.
23. The ocular implant according to any embodiment 1 to 19 wherein the composition does not contain any release modulating agent
24. The ocular implant according to any preceding embodiment, wherein the at least one coating layer is porous.
25. The ocular implant according to any preceding embodiment, wherein the at least one coating layer has a thickness between 1 and 150 μm.
26. The ocular implant according to any preceding embodiment, wherein the at least one layer further comprises the therapeutic ingredient or an additional therapeutic ingredient.
27. The ocular implant according to any preceding embodiment, which is a macro, micro or nanoparticle.
28. The ocular implant according to any preceding embodiment, wherein the therapeutic agent is present in a concentration of 200 μg and 2000 μg per μm³ of ocular implant.
29. A method of making an ocular implant of any embodiment 1 to 28, comprising the steps of:
   a) Providing the therapeutic agent;
   b) Obtaining an ocular composition by mixing the therapeutic agent with the polymerizable composition, the biodegradable polymer, a photoinitiator and optionally the release modulating agent;
   c) Irradiating the ocular composition obtained under step b) with light at a wavelength between 200 and 550 nm for a period of time between 1 second and 60 minutes to form an uncoated ocular implant;
   d) Coating at least a portion of the uncoated ocular implant external surface with at least one coating layer.

30. The method of embodiment 29, wherein the therapeutic agent is first dissolved into a solvent to obtain a solution before the so obtained solution is mixed with the polymerizable composition, the biodegradable polymer, the photoinitiator and optionally the release modulating agent.
31. The method of embodiment 29 or 30, wherein the photoinitiator is a hydroxyketone photoinitiator, an amino ketone photoinitiator, a hydroxy ketone/benzophenone photoinitiator, a benzyldimethyl ketal photoinitiator, a phenylglyoxylate photoinitiator, an acylphosphine oxide photoinitiator, an acyl phosphine oxide/alpha hydroxy ketone photoinitiator, a benzophenone photoinitiator, a ribityl isoalloxazine photoinitiator, or a phenyglyoxylate photoinitiator or any combination thereof.
32. The method of embodiment 31, wherein the photoinitiator is 1-[4-(2-hydroxyethoxy)-phenyl]-2-hydroxy-2-methyl-1-propanone, 2,2-dimethoxy-2-phenylacetophenone (DMPA) or 2-Hydroxy-1-[4-(2-hydroxyethoxy) phenyl]-2-methyl-1-propanone (Irgacure 2959) or riboflavin.
33. The method of any embodiment 29 to 32, wherein under step d) the uncoated ocular implant is coated on the totality of its external surface with at least one coating layer.
34. The method of any embodiment 29 to 33, wherein the step d) of coating is performed by manual dipping, controlled dip-coating ultrasound coating, spray coating or 3D printing.
35. A method of making an ocular implant of any embodiment 1 to 28, comprising the steps of:
   a) Providing the therapeutic agent;
   b) Obtaining an ocular composition by mixing the therapeutic agent with the polymerizable composition, the biodegradable polymer, a photoinitiator and optionally the release modulating agent;
   c) Injecting the ocular composition obtained under step b) into a preformed hollow coating layer;
   d) Irradiating the ocular composition within the hollow coating layer with light at a wavelength between 200 and 550 nm for a period of time between 1 second and 60 minutes.
36. The method of embodiment 35, wherein the hollow coating layer is a hollow tube.

The following examples serve to illustrate the invention, however, should not to be understood as restricting the scope of the invention.

### EXAMPLES

### Example 1

### Dexamethasone (DEX) and Timolol Maleate (TM) with or without poly(L-lactide) PLA Coating

### 1.1. Materials

Poly(ethylene glycol) diacrylate (Mn=700, PEGDA 700), poly(ethylene glycol) diacrylate (Mn=250, PEGDA 250), dichloromethane, sodium hydroxide (NaOH), Irgacure 2959, N-Methyl-2-pyrrolidone (NMP) and acetonitrile were purchased from Sigma (Dorset, UK). Dexamethasone (DEX) was bought from Bufa (Hilversum, the Netherlands). Poly(lactide-co-glycolide) (PURASORB® PDLG 5002, 50:50, PLGA 50/50), poly(lactide-co-glycolide) (PURASORB® PDLG 7502, 75:25, PLGA 75/25) and poly(L-lactide) (PURASORB® PL 65, PLA) were obtained from Purac Biochem (Gorinchem, The Netherlands), Timolol Maleate from Gangwal Chemicals Pvt Ltd (Maharashtra, India).

1.2. Preparation of Rod Shape Implants for DEX (DEX 10% w/w, PLGA 20% w/w, PEGDA 700 70% w/w)

PEGDA 700 (280 mg), PLGA 50/50 (80 mg) and DEX (40 mg) were mixed and stirred overnight. 90 μL of photoinitiator solution (40 mg/mL solution of Irgacure 2959 in pure ethanol) was added and the mixture was stirred for 10 min. The resultant mixture was injected into silicone tubes and photo-crosslinked using a light hammer (Light Hammer® 6, Heraeus Noblelight Fusion UV Inc., Gaithersburg Md., USA). The intensity of the UV light was set as 100% and the silicone tubes were exposed to the UV light for 30 sec (10 runs, 5 runs on each side). Then the rod shape implants were removed from the tubes. To prepare coated implants, the uncoated implants were dipped into PLA solution (2.5% PLA in dichloromethane) for 3 sec and then left dry in the fume hood for 48 h.

1.3. Preparation of Rod Shape Implants for TM (TM 10% w/w, PLGA 75/25 20% w/w, PEGDA 250 70% w/w)

TM (20 mg) was first dissolved in NMP (30 μL), and then mixed with PEGDA 250 (140 mg) and PLGA 75/25 (40 mg). The mixture was stirred overnight 45 μL of photoinitiator solution (40 mg/mL solution of Irgacure 2959 in ethanol) was added and the mixture was stirred for 10 min. The resultant mixture was injected into silicone tubes and photo-crosslinked using a light hammer (Light Hammer® 6, Heraeus Noblelight Fusion UV Inc., Gaithersburg, Md., USA). The intensity of the UV light was set as 100% and the silicone tubes were exposed to the UV light for 30 sec (10 runs). Then the rod shape implants were removed from the tubes. To prepare coated implants, the uncoated implants were dipped into PLA solution (2.5% PLA in dichloromethane) for 3 sec and then left dry in the fume hood for 48 h.

### 1.4. Determination of DEX Using High-Performance Liquid Chromatography (HPLC)

DEX was determined by reverse-phase HPLC. The HPLC instrument consisted of Agilent 1260 Infinity pump equipped with a sample injection port fitted with 20 μl sample loop, a UV-VIS detector and a Chromato-Integrator (Agilent Technologies, Germany). The mobile phase consisted of acetonitrile and water in the ratio 40:60. The flow rate of mobile phase was 0.8 mL/min and the eluted drug was detected at 245 nm wavelength. Chromatographic separation of the DEX was achieved at ambient room temperature (24±2° C.) using Poroshell 120 EC-C18 4 μm (250×4.60 mm) analytical column fitted with a refillable guard column. The mobile phase was filtered by passing through 0.45 μm membrane filter (Whatman International, UK) under vacuum and degassed before use.

### 1.5. Determination of TM Using High-Performance Liquid Chromatography (HPLC)

TM content was determined by reverse-phase HPLC. The HPLC instrument consisted of Agilent 1260 Infinity pump equipped with a sample injection port fitted with 20 μl sample loop, a UV-VIS detector and a Chromato-Integrator (Agilent Technologies, Germany). The mobile phase consisted of acetonitrile (0.05% v/v TFA) and water (0.05% v/v TFA) in the ratio 40:60. The flow rate of mobile phase was 0.8 mL/min and the eluted drug was detected at 295 nm wavelength. Chromatographic separation of the TM was achieved at ambient room temperature (24±2° C.) using Poroshell 120 EC-C18 4 μm (250×4.60 mm) analytical column fitted with a refillable guard column. The mobile phase was filtered by passing through 0.45 μm membrane filter (Whatman International, UK) under vacuum and degassed before use.

### 1.6. In Vitro Drug Release Studies

The drug-loaded implants (4 mg, diameter 0.635 mm, length 10 mm) were immersed in 20 mL PBS (pH=7.4) and kept in a horizontal shaking incubator at 37° C. and 40 rpm. The drug release supernatant (1.7 mL) was collected periodically (24, 48, 72 h, etc.) and replaced with fresh medium. The drug content in the aliquots was determined by HPLC. All release experiments were carried out in 3-fold, and all data were averages of three determinations. Table 1 Summarizes the parameters for Implants DEX 1, DEX 2, TM 1, TM 2

| | Therapeutic | | | |
|---|---|---|---|---|
| | Agent | PLGA | PEGDA | PLA |
| Formulation | (w/w %) | (w/w %) | (w/w %) | Coating |
| DEX 1 | 10% DEX | 20%, 50/50 | 70% 700 | — |
| DEX 2 | 10% DEX | 20%, 50/50 | 70% 700 | 1 layer |
| TM 1 | 10% TM | 20%, 75/25 | 70% 250 | — |
| TM 2 | 10% TM | 20%, 75/25 | 70% 250 | 3 layers |

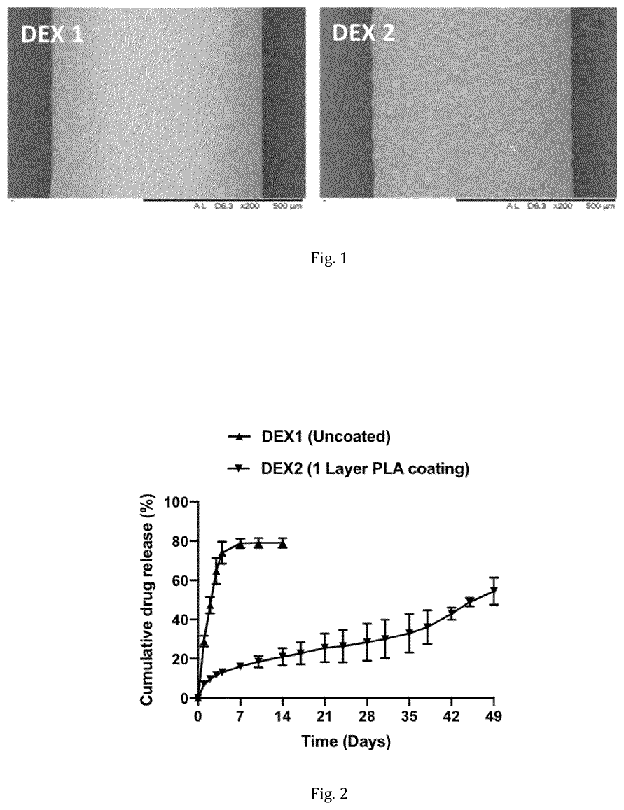

Surface morphology of the implants were characterized by SEM, as shown in FIG. 1. DEX 2 has a slightly rough surface while DEX 1 appears to have a smooth surface. The diameter of the rod shape implants is approximately 0.635 mm. The thickness of the coating is approximately 0.029 mm, i.e. 29 μm.

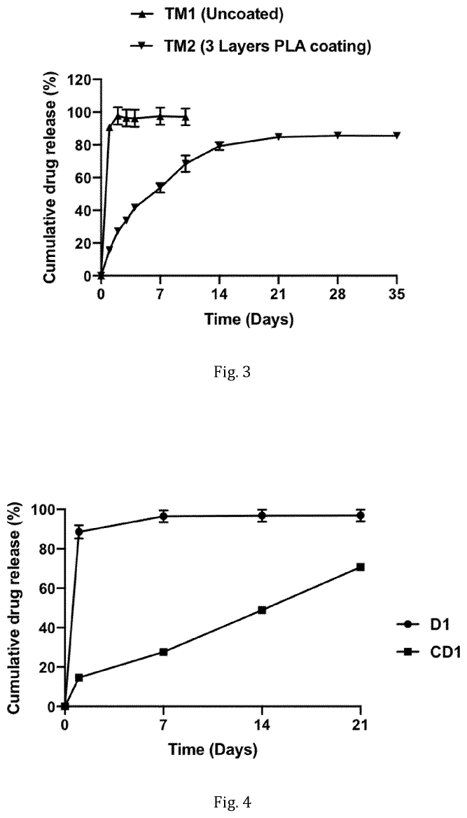

As can be seen from FIGS. 2 and 3, comparative implants DEX 1 and TM 1 show a considerable burst release on the first day. This effect is greatly suppressed in DEX 2 and TM 2. The implants according to the invention can provide a sustainable release of the therapeutic agent over a prolonged period of time.

### Example 2

### Fluorescein isothiocyanate (FITC)-Dextran Implants with & without poly-L-lactide-co-caprolactone (PLC) and poly (DL-Lactide) (PDL) Coating

| | FITC-Dextran | PLGA | PEGDA | Dimensions |
|---|---|---|---|---|
| | (4 kDa) loading | 75/25 | 700 | (D * L) |
| Formulation | (w/w %) | (w/w %) | (w/w %) | mm |
| D1 | 10 | 5 | 85 | 0.5 * 7.5 |
| CD1 (Coated) | 10 | 5 | 85 | 0.5 * 7.5 |

### 2.1. Preparation of D1

10 mg of PLGA 75/25 (Purac Biochem, Gorinchem, The Netherlands) was dissolved in 190 mg of PEGDA of molecular weight (MW) 700 Da (Sigma Aldrich, Basingstoke, England) to prepare Solution A. 5 mg Irgacure 2559 (Sigma Aldrich, Basingstoke, England) was dissolved in 1 ml PBS to prepare Solution B. 10 mg of FITC dextran (average MW 4000 Da, Sigma Aldrich. Basingstoke, England) was dissolved into a 60 μl of Solution B in an Eppendorf tube to prepare Solution C. 85 mg of Solution A was weighed in an empty Eppendorf tube and 60 μl of Solution C was added to the mixture slowly through Eppendorf tube wall with continuous stirring at 900 rpm for 15 minutes. The mixtures finally obtained was withdrawn into silicon tubes with ID of 0.635 mm (Polymer System Technology, England) and cross-linked using a UV light (Light Hammer® 6, Heraeus Noblelight Fusion UV Inc., Gaithersburg, Md., USA). The intensity of the UV light was set as 50% and the silicone tubes were exposed to the UV light for 15 seconds (i.e. a total of 5 runs). The implants were then removed from the silicon tubes and left to dry in vacuum at 25° C. for 4 hours. The rod-shaped implants were cut at each 7.5 mm length.

2.2. Preparation of CD1 (Coated with PLC)

Implants CD1 were manufactured according to Section 2.1 as described above (except last sentence). They were cut into 20 mm length and coated with 17% w/v solution of poly-L-lactide-co-caprolactone (PLC 8516) (Purac Biochem, Gorinchem, The Netherlands) in dichloromethane (DCM) using a texture analyser instrument (TA-XT plus; Stable Micro Systems, US). The implant was dipped at speed of 10 mm/s, held for 1 s inside the coating solution, then withdrawn at speed of 10 mm/s. A single coat layer was applied with thickness of about 20-25 μm. The implants were then cut into 7.5 mm length and the sides of these surface-coated implants were coated with 15% w/v poly-DL-lactide (PDL) solution in acetonitrile (ACN) by using a 29G needle syringe under digital microscope.

### 2.3 In Vitro Drug Release Set Up

Two implants of D1 and two implants of CD1 (of 7.5 mm length) were placed into two glass vials containing 2 mL of PBS (Phosphate buffered saline) with 0.01% w/v Sodium azide (NaN₂) (pH 7.4±0.2) as release media. All the experiments were carried out in triplicate. The glass vials containing the implants were placed in a shaking orbital incubator at a speed of 40 rpm and at 37° C. (GFL Orbital Shaking Incubator; Gesellschaft für Labortechnik mbH, Germany). Sampling followed by complete replacement of the PBS medium was performed on Day 1 and weekly thereafter, i.e. Day 7, Day 14, Day 21, Day 28 and so on. The concentration of released drug molecule in the PBS samples was analyzed as described in the following section. The vials were then incubated at 37° C. and at predetermined time intervals the entire medium was removed and replaced with fresh medium.

### 2.4 Sample Analysis

Analysis of FITC-dextran in vitro drug release samples were performed using the fluorescence spectrophotometry method. Detection was carried out by micro 96 well plate spectrophotometer (BMG Labtech FLUOstar Optima fluorescence plate reader (BMG Labtech GmbH, Ortenberg, Germany). Excitation was set to 485 nm, emission was set to 520 nm, and gain was set to 750.

FIG. 4 shows the in vitro release of D1 and CD1 expressed as percentage cumulative release. As it can be seen from this figure, the presence of the coating polymer layer on the implant matrix significantly reduces the burst effect and the overall release of FITC-dextran is controlled over the entire period of time.

### Example 3

### Latanoprost (LP) Implants with Different Diameter Size and with or without poly-L-lactide-co-caprolactone (PLC) Coating

| | Latanoprost | PLGA | PEGDA | Dimensions |
|---|---|---|---|---|
| | loading | 75/25 | 250 | (D * L) |
| Formulation | (w/w %) | (w/w %) | (w/w %) | mm |
| LP1 | 20 | 30 | 50 | 0.3 * 2 |
| LP2 | 20 | 30 | 50 | 0.6 * 2 |
| LPC1 | 20 | 30 | 50 | 0.3 * 2 |
| (1 layer coated) | | | | |
| LPC2 | 20 | 30 | 50 | 0.6 * 2 |
| (1 layer coated) | | | | |

### 3.1. Preparation of LP1 and LP2

20 mg Irgacure 2959 (Sigma Aldrich, Basingstoke, England) was dissolved in acetonitrile to prepare Solution A. 50 mg Latanoprost (LP) (Alfa Chemistry, New York, USA) was dissolved in 2.5 mL acetonitrile to prepare Solution B. 75 mg PEGDA 250 and 15 mg of PLGA 75/25 (Purac Biochem, Gorinchem, The Netherlands) were put into a 2 mL Eppendorf tube, and dissolved in 250 μL acetonitrile to prepare Solution C.

37.5 μL of Solution A and 1 mL of Solution B were then added to Solution C, and subsequently stirred at 250 rpm for 30 minutes (Multistirrer, Velp Scientifica™, Italy). Acetonitrile was then evaporated under gauge pressure of −0.1 MPa at room temperature for 6 h (OV-12 vacuum oven; JeioTech, Korea). The mixture finally obtained was withdrawn into a silicon tube of internal diameter 0.32 or 0.63 mm (HelixMark® Standard Silicone Tubing; Freudenberg, Germany) by using a 25 G needle attached to 10 mL syringe. Photocrosslinking was performed for 10 runs under UV D-lamp operated at 100% intensity with a belt speed of 11.5 m/min (Light Hammer 6; Heraeus Noblelight Fusion UV, USA). The solidified rod-shaped implant was removed from the silicone tubing and cut into a 2 mm length. The implants had a weight of about 0.2 mg (for 0.3 mm diameter, LP1) and 0.9 mg (for 0.6 mm diameter, LP2).

3.2 Preparation of LPC1 and LPC2 (LP1 and LP2 Coated with PLC)

Implants LP1 and LP2 were coated by automated dip coating method to obtain LPC1 and LPC2, respectively. A single coat layer was applied with thickness of about 20-25 μm. They were coated on surface with 17% w/v solution of poly-L-lactide-co-caprolactone (PLC) (Purac Biochem, Gorinchem, The Netherlands) polymer solution in dichloromethane (DCM) using Texture analyser instrument and on sides with 15% w/v poly-DL-lactide (PDL) (Purac Biochem, Gorinchem, The Netherlands) solution in acetonitrile (ACN) by using a 29 G needle syringe under digital microscope.

### 3.3 In Vitro Drug Release Set Up

Implants LP1, LP2, LPC1 and LPC2 were each placed in a centrifuge tube containing 2 mL of PBS (Phosphate buffered saline) with 0.01% w/v Sodium azide (NaN₂) (pH 7.4±0.2) as release media. All the experiments were carried out in triplicate. The centrifuge tubes containing implants were placed in a shaking orbital incubator at a speed of 40 rpm and at 37° C. (GFL Orbital Shaking Incubator; Gesellschaft für Labortechnik mbH, Germany). Sampling followed by complete replacement of the PBS medium was performed on Day 1, Day 3, Day 7 and weekly thereafter. The concentration of released drug was analysed using a developed HPLC method for Latanoprost

### 3.4 Sample Analysis

Analysis of LP1, LP2, LPC1 and LPC2 samples was performed using HPLC system with fluorescence detection (Agilent 1260 Infinity II Quaternary System) using a Poroshell 120 EC-C18 column (250 mm length, 4.6 mm internal diameter and 4 μm particle size). The samples were analyzed in an isocratic mode using a mobile phase of acetonitrile: 0.1% v/v formic acid (60:40), with an injection volume of 50 μL and a flow rate of 1 mL/min. The column temperature was maintained at 40° C. The fluorescence detector was set at an excitation wavelength of 265 nm and an emission wavelength of 285 nm.

FIG. 5 shows the in vitro release of LP1, LP2, LPC1 and LPC2 expressed as percentage cumulative. As it can be seen from the figure, the presence of the coating polymer layer on the implant matrix significantly reduces the burst effect and the overall release of LP is controlled over the entire period of time.

### Example 4

### Latanoprost (LP) Implants with One or More Layers of poly-L-lactide-co-caprolactone (PLC)—Effect of the Layers

| | Latanoprost | PLGA | PEGDA | Dimensions |
|---|---|---|---|---|
| | loading | 75/25 | 250 | (D * L) |
| Formulation | (w/w %) | (w/w %) | (w/w %) | mm |
| LPC1 | 20 | 30 | 50 | 0.3 * 2 |
| (1 layer coated) | | | | |
| LPC3 | 20 | 30 | 50 | 0.3 * 2 |
| (2 layers coated) | | | | |

### 4.1. Preparation of LP3

LPC3 implants were prepared from LP1 implants using the coating method described under Section 3.2, whereby the automated dip coating was repeated a second time on dried LPC1 implants to achieve 2 layers of PLC coating.

### 4.2 In Vitro Drug Release Set Up and Sample Analysis

A LPC1 and a LPC3 implant, 2 mm long and having a weight of about 0.2 mg were each placed in a centrifuge tube containing 2 mL of PBS (Phosphate buffered saline) with 0.01% w/v Sodium azide (NaN₂) (pH 7.4±0.2) as release media. All the experiments were carried out in triplicate. The centrifuge tubes containing implants were placed in a shaking orbital incubator at a speed of 40 rpm and at 37° C. (GFL Orbital Shaking Incubator; Gesellschaft für Labortechnik mbH, Germany). Sampling followed by complete replacement of the PBS medium was performed on Day 1, Day 3, Day 7 and weekly thereafter. The concentration of released drug was analyzed using a developed HPLC method for latanoprost.

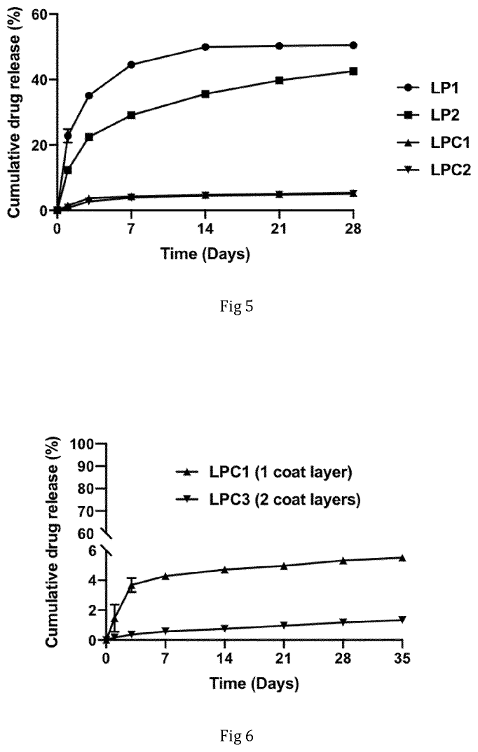

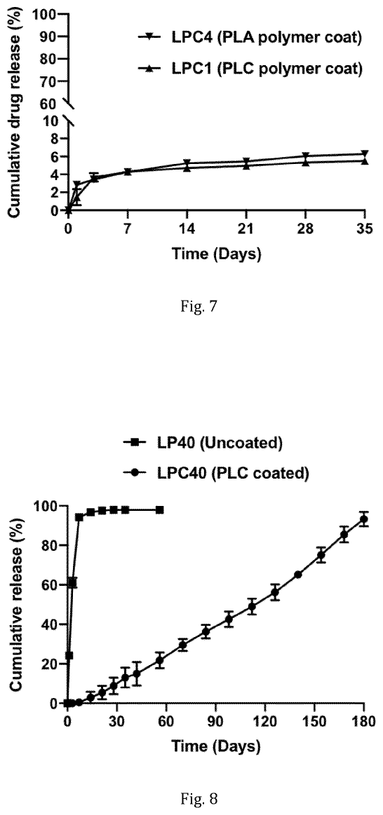

FIG. 6 shows the in vitro release of LPC1 and LPC3 expressed as percentage cumulative. As it can be seen from the figure, an additional coating polymer layer on the implant matrix further reduces the burst effect and the overall release of latanoprost is controlled over the entire period of time.

### Example 5

### Latanoprost (LP) Implants Coated with Layers of poly-L-lactide-co-caprolactone (PLC) and poly(L-lactide) (PLA)—Effect of the Composition of the Coating Material

| | Latanoprost | PLGA | PEGDA | Dimensions |
|---|---|---|---|---|
| | loading | 75/25 | 250 | (D * L) |
| Formulation | (w/w %) | (w/w %) | (w/w %) | mm |
| LPC1 | 20 | 30 | 50 | 0.3 * 2 |
| (PLC coated) | | | | |
| LPC4 | 20 | 30 | 50 | 0.3 * 2 |
| (PLA coated) | | | | |

### 5.1. Preparation of LPC4

LPC4 implants were prepared by coating LP1 implants by automated dip coating method. The LPC4 implants were coated on surface with 2.5% w/v solution of poly(L-lactide) (PLA) polymer solution in dichloromethane (DCM) using Texture analyzer instrument and on sides with 15% w/v poly(DL-lactide) (PDL) (Purac Biochem, Gorinchem, The Netherlands) solution in acetonitrile (ACN) by using a 29 G needle syringe under digital microscope.

### 5.2 In Vitro Drug Release Set Up and Sample Analysis

A LPC1 and a LPC4 implant, 2 mm long and having a weight of about 0.2 mg were each placed in a centrifuge tube containing 2 mL of PBS (Phosphate buffered saline) with 0.01% w/v Sodium azide (NaN₂) (pH 7.4±0.2) as release media. All the experiments were carried out in triplicate. The centrifuge tubes containing implants were placed in a shaking orbital incubator at a speed of 40 rpm and at 37° C. (GFL Orbital Shaking Incubator; Gesellschaft für Labortechnik mbH, Germany). Sampling followed by complete replacement of the PBS medium was performed on Day 1, Day 3, Day 7 and weekly thereafter. The concentration of released drug was analyzed using a developed HPLC method for latanoprost.

FIG. 4 shows the in vitro release of LPC1 and LPC4 expressed as percentage cumulative. As it can be seen from these figures, both coating polymer materials reduce the burst effect (compared to LP1) and the overall release of latanoprost is controlled over the entire period of time.

### Example 6

### High Loading Latanoprost (LP) Implants with or without PLC Coating

| | Latanoprost | PLGA | PEGDA | Dimensions |
|---|---|---|---|---|
| | loading | 75/25 | 250 | (D * L) |
| Formulation | (w/w %) | (w/w %) | (w/w %) | mm |
| LP40 | 40 | 2 | 58 | 0.3 * 2 |
| LPC40 | 40 | 2 | 58 | 0.3 * 2 |
| (1 layer coated | | | | |
| PLC) | | | | |

### 6.1. Preparation of LP40

20 mg Irgacure 2959 (Sigma Aldrich, Basingstoke, England) was dissolved in acetonitrile to prepare Solution A. 50 mg Latanoprost (LP) (Alfa Chemistry, New York, USA) was dissolved in 2.5 mL acetonitrile to prepare Solution B. 29 mg PEGDA 250 and 1 mg PLGA 75/25 (Purac Biochem, Gorinchem, The Netherlands) were put into a 2 mL Eppendorf tube, and dissolved in 250 μL acetonitrile to prepare Solution C.

14.5 μL of Solution A and 1000 μL of Solution B were then added to Solution C, and subsequently stirred at 250 rpm for 30 minutes (Multistirrer, Velp Scientifica™, Italy). Acetonitrile was then evaporated under gauge pressure of −0.1 MPa at room temperature for 6 h (OV-12 vacuum oven; JeioTech, Korea). The mixture finally obtained was withdrawn into a silicon tube of internal diameter 0.32 (HelixMark® Standard Silicone Tubing; Freudenberg, Germany) by using a 25 G needle attached to 10 mL syringe. Photocrosslinking was performed for 5 runs under UV D-lamp operated at 50% intensity with a belt speed of 11.5 m/min (Light Hammer 6; Heraeus Noblelight Fusion UV, USA). The solidified rod-shaped implant was removed from the silicone tubing and cut into a 2 mm length. The implants had a weight of about 0.2 mg.

### 6.2 Preparation of LPC40

Implants LP40 were coated by automated dip coating method to obtain LPC40. A single coat layer was applied with thickness of around 20-25 μm. They were coated on surface with 17% w/v solution of poly-L-lactide-co-caprolactone (PLC) (Purac Biochem, Gorinchem, The Netherlands) polymer solution in dichloromethane (DCM) using Texture analyser instrument and on sides with 15% w/v poly-DL-lactide (PDL) (Purac Biochem, Gorinchem, The Netherlands) solution in acetonitrile (ACN) by using a 29 G needle syringe under digital microscope.

### 6.3 In Vitro Drug Release Set Up and Sample Analysis

A LP40 and a LPC40 implant of 2 mm length and having a weight of about 0.2 mg were each placed in a centrifuge tube containing 2 mL of PBS (Phosphate buffered saline) with 0.01% w/v Sodium azide (NaN₂) (pH 7.4±0.2) as release media. All the experiments were carried out in triplicate. The centrifuge tubes containing implants were placed in a shaking orbital incubator at a speed of 40 rpm and at 37° C. (GFL Orbital Shaking Incubator; Gesellschaft für Labortechnik mbH, Germany). Sampling followed by complete replacement of the PBS medium was performed on Day 1, Day 3, Day 7 and weekly thereafter. The concentration of released drug was analyzed using a developed HPLC method for latanoprost

FIG. 8 shows the in vitro release of LP40 & LPC40 expressed as percentage cumulative release. As it can be seen from this figure, coating on the surface of implants significantly reduced initial burst release and sustained the release over longer period as compared to uncoated implants. The coated LPC40 implants maintained near zero-order release for 180 days (6-months) while uncoated LP40 implants could achieve sustained release for 20 days.

1. An ocular implant comprising:
a) at least 0.1% w/w of a therapeutic agent;
b) 5 to 95% w/w of a crosslinked polymer matrix;
c) and 0.1 to 40% w/w of a biodegradable polymer selected from the group consisting of lactide/glycolide copolymer (including poly(lactide-co-glycolide) (PLGA)), poly (L-lactide) (PLA), polyhydroxyalkanoates, including polyhydroxybutyrate, polyglycolic acid (PGA), polycaprolactone (PCL), poly (DL-lactide) (PDL), poly (D-lactide), lactide/caprolactone copolymer, poly-L-lactide-co-caprolactone (PLC) and mixtures, copolymers, and block copolymers thereof;
wherein the crosslinked polymer matrix is obtained by crosslinking a photopolymerizable composition selected from the group consisting of fragments or monomers of polyalkylene glycol mono-acrylate, polyalkylene glycol diacrylate, polyalkylene glycol mono-methacrylate and polyalkylene glycol dimethacrylate, and mixtures, copolymers, and block copolymers thereof,
characterized in that the ocular implant is at least partially coated on its external surface with at least one coating layer selected from the group consisting of lactide/glycolide copolymer (including poly(lactide-co-glycolide) (PLGA)), poly (L-lactide) (PLA), polyhydroxyalkanoates, including polyhydroxybutyrate, polyglycolic acid (PGA), polycaprolactone (PCL), lactide/caprolactone copolymer, poly (DL-lactide) (PDL), poly (D-lactide), poly-L-lactide-co-caprolactone (PLC) and mixtures, copolymers, and block copolymers thereof; crosslinked fragments or monomers of polyalkylene glycol mono-acrylate, polyalkylene glycol diacrylate, polyalkylene glycol methacrylate and polyalkylene glycol dimethacrylate, and mixtures, copolymers, and block copolymers thereof.

2. The ocular implant according to claim 1, wherein the therapeutic agent is present in an amount between 0.5 and 70% w/w.

3. The ocular implant according to claim 2, wherein the therapeutic agent is present in an amount between 10 and 50% w/w.

4. The ocular implant according to claim 1, wherein the photopolymerizable composition is selected from the group consisting of polyethylene glycol diacrylate, diethylene glycol diacrylate, polyethylene glycol dimethacrylate, diethylene glycol dimethacrylate, polypropylene glycol diacrylate, dipropylene glycol diacrylate, dipropylene glycol dimethacrylate, and polypropylene glycol dimethacrylate.

5. The ocular implant according to claim 4, wherein the photopolymerizable composition is polyethylene glycol diacrylate (PEGDA).

6. The ocular implant according to claim 1, wherein the biodegradable polymer is present in an amount between 1 and 30% (w/w).

7. The ocular implant according to claim 1, wherein the biodegradable polymer is lactide/glycolide copolymer, including poly(lactide-co-glycolide) (PLGA).

8. The ocular implant according to claim 1, wherein the at least one coating layer is poly (L-lactide) (PLA), poly (DL-lactide) (PDL), poly-L-lactide-co-caprolactone (PLC) and combinations thereof.

9. The ocular implant according to claim 8, wherein the at least one coating layer is poly-L-lactide-co-caprolactone (PLC), poly (L-lactide) (PLA) or mixtures thereof.

10. The ocular implant according to claim 1, wherein the implant is coated on the totality of its external surface with at least one coating layer.

11. The ocular implant according to claim 1, having a first and a second portion of external surface, wherein the first and second portion of the external surface are each coated with at least one coating layer independently selected from the group consisting of lactide/glycolide copolymer (including poly(lactide-co-glycolide) (PLGA)), poly (L-lactide) (PLA), polyhydroxyalkanoates, including polyhydroxybutyrate, polyglycolic acid (PGA), polycaprolactone (PCL), lactide/caprolactone copolymer, poly (DL-lactide) (PDL), poly (D-lactide), poly-L-lactide-co-caprolactone (PLC) and mixtures, copolymers, and block copolymers thereof; crosslinked fragments or monomers of polyalkylene glycol mono-acrylate, polyalkylene glycol diacrylate, polyalkylene glycol methacrylate and polyalkylene glycol dimethacrylate, and mixtures, copolymers, and block copolymers thereof.

12. The ocular implant according to claim 1, further comprising a release modulating agent, preferably selected from polyethylene glycol, hydroxypropyl methylcellulose (HPMC), maltose, glucose, agarose, mannitol, gelatin, sodium chloride, magnesium carbonate, magnesium hydroxide, potassium chloride, sodium bicarbonate, potassium bicarbonate and sucrose.

13. The ocular implant according to claim 1, wherein the at least one coating layer is porous.

14. A method of making an ocular implant of claim 1, comprising the steps of:
a) providing the therapeutic agent;
b) obtaining an ocular composition by mixing the therapeutic agent with the polymerizable composition, the biodegradable polymer, a photoinitiator and optionally the release modulating agent;
c) irradiating the ocular composition obtained under step b) with light at a wavelength between 200 and 550 nm for a period of time between 1 second and 60 minutes to form an uncoated ocular implant;
d) coating at least a portion of the uncoated ocular implant external surface with at least one coating layer.

15. A method of making an ocular implant of claim 1, comprising the steps of:
a) providing the therapeutic agent;
b) obtaining an ocular composition by mixing the therapeutic agent with the polymerizable composition, the biodegradable polymer, a photoinitiator and optionally the release modulating agent;
c) injecting the ocular composition obtained under step b) into a preformed hollow coating layer
d) irradiating the ocular composition within the hollow coating layer with light at a wavelength between 200 and 550 nm for a period of time between 1 second and 60 minutes.
